(19) United States
(12) Patent Application Publication
(10) Pub. No.: US 2021/0315535 A1
(43) Pub. Date: Oct. 14, 2021

(54) SYSTEM AND METHOD FOR USE IN X-RAY IMAGING

(71) Applicant: LensFree Ltd., Ra'anana (IL)

(72) Inventors: Zeev ZALEVSKY, Rosh HaAyin (IL); Doron AVRAHAM, Petach Tikva (IL); Yossi DANAN, Rehovot (IL)

(21) Appl. No.: 17/287,583

(22) PCT Filed: Oct. 23, 2019
(86) PCT No.: PCT/IL2019/051141

§ 371 Date: Apr. 22, 2021

Related U.S. Application Data
(60) Provisional application No. 62/749,178, filed on Oct. 23, 2018.

(51) Int. Cl. A61B 6/00

(52) U.S. Cl. CPC A61B 6/54; A61B 6/4291; A61B 6/4007

(57) ABSTRACT

An imaging system and corresponding imaging method are described. The system comprising: a radiation emitting unit, configured for emitting electromagnetic radiation of a selected wavelength range and a detector array located at a selected image plane located downstream of said aperture plane. The radiation emitting unit comprises a plurality of radiation emitting element are configured for focusing said radiation onto corresponding selected arrangement of focusing spot on an aperture plane of the imaging system. The imaging system is configured for imaging an object located between said plurality of radiation emitting elements and said aperture plane.

### TECHNOLOGICAL FIELD

The invention is in the field of x-ray radiation imaging systems and more specifically relates to imaging systems utilizing pinhole array(s) and focused radiation emission.

### BACKGROUND

The use of pinhole optics in imaging systems is generally known. The basic principles of a pinhole-based imaging system (e.g. pinhole camera) relate to direction of radiation/light rays arriving from one point in the object toward a common location on an image plane. This enables imaging while avoiding the use of refractive lens(es), which is replaced by a small aperture. More specifically, light arriving from an object passes through the aperture (small pinhole) and projects an inverted image of the region of interest (the object) on the opposite side of the imaging system. This is also known the “camera obscura” effect.

Pinhole optics provides advantages over traditional common lens-based optical systems such as reducing linear distortion, providing virtually infinite depth of focus and wide angular field of view. Additionally, pinhole imaging is useful for non-optical radiation frequencies, such as X-rays, Gamma radiation and basically any wave- or particle-like phenomena.

These advantages typically come with price of reduced brightness associated with small diameter of the aperture as compared with collection area of a lens. However, recently additional imaging techniques enable the use of a plurality of pinholes enabling imaging with increased energetic efficiency and proper image restoration using a selected set of pinhole arrays having suitable arrangement avoiding loss of data that may result from interference of radiation passing through the different pinholes of each array.

### GENERAL DESCRIPTION

Pinhole based imaging enables energetically efficient imaging in non-optical electromagnetic frequencies such as X-ray, gamma or other non-optical radiation fields. The present technique utilizes concepts of pinhole imaging while allowing further increased energetic efficiency by directing emitted radiation to be converged at an aperture plane associated with location of one or more pinholes. According to the present technique, the use of such structured radiation emission allows efficient pinhole-based imaging while omitting the need for actual mask having pinholes. However, such mask may be used in some configurations for optimization of signal to noise ratio.

To this end, the present technique provides an imaging system comprising an arrangement of a plurality of radiation emitting elements configured for directing electromagnetic radiation of one or more selected wavelength range toward a general direction where an object is located, and a detector array located downstream with respect to said general direction and is configured for collecting radiation impinging thereon for generating image data associated with said object. The radiation emitting elements are configured for emitting said electromagnetic radiation in a converging path for generating corresponding focusing spots at a selected aperture plane of the imaging system. Said aperture plane is located between said object and said detector array.

Effectively, this configuration directs high intensity portion of the emitted radiation toward one or more selected spots on the aperture plane, acting at one or more pinholes with respect to radiation propagation. This arrangement of the radiation emitting elements allows collection of directly transmitted radiation passing through the one or more focusing spots (acting as pinholes), enabling increased efficiency as compared to shaded imaging as conventionally used, as well as the collection of scattered radiation associated with conventional pinhole-based imaging.

The plurality of radiation emitting elements may include selected groups of radiation emitting elements positioned and oriented for focusing emitted radiation toward selected corresponding spots on the aperture plane. More specifically, the plurality of radiation emitting elements may include plurality of two or more groups of radiation emitting elements, where radiation emitting elements of each group are oriented to direct emitted radiation toward a common spot on the aperture plane. Further, the plurality of two or more groups may comprise two or more sets of groups, where groups of each set direct emitted radiation toward a selected array of spots forming an effective aperture array on the aperture plane.

This configuration may provide radiation emission directed toward a selected set of focusing spots on the aperture plane. The radiation propagates from the aperture plane toward the detector array, which is located at an image plane. The radiation forms a plurality of image replications corresponding with the plurality of focusing spots. Generally, the arrangement of focusing spots associated with each set of radiation emitting elements acts as an array of apertures, comprising one or more apertures having selected spatial arrangement. Collection of image data formed by two or more sets (two or more arrays of focusing spots) enables image reconstruction of the object with selected brightness (energetic efficiency). The two or more sets of radiation emitting elements are directed toward corresponding two or more sets of arrays of focusing spots, each having corresponding transmission function. Generally, the different arrays of focusing spots may be selected such that total spatial transmission function of the two or more arrays have non-null transmission for spatial frequencies up to a selected maximal spatial frequency.

According to some embodiments, the imaging system may also comprise, or be connected with a control unit configured for selectively operating the plurality of radiation emitting elements, and for receiving collected image data from the detector array. Generally, the control unit may operate the radiation emitting elements by starting emission from emitting elements of one set for a selected emission time, then stop emission from the first set and start emission from radiation emitting elements of another set for a corresponding selected emission time, to provide emission from the plurality of two or more sets, each with corresponding emission time. The detector array may provide image data associated with radiation from each set of radiation emitting elements separately, or measure impinging radiation from the different sets of radiation emitting elements and provide combined output image data. In some embodiments, the control unit may utilize data on arrangement of the two or more focusing spots associated with the two or more sets for use in reconstructions of the image data to provide data about the object being imaged.

Generally, the radiation emitting elements used in the present technique are configured for emitting electromagnetic radiation of one or more selected wavelength ranges, and for directing the emitted radiation toward corresponding focusing spot located at the aperture plane. To this end, the radiation emitting elements are configured for manipulating emitted radiation for directing at least a portion of the emitted radiation in converging path toward the focusing spot. Generally, high energy radiation such as UV and W-ray radiation is considered as non-optical radiation, i.e. the use of refractive lens for manipulating the radiation is challenging. To this end the present invention utilizes diffractive and interference effects for focusing the emitted radiation, as well as the use of structure emission pattern in certain configurations. The diffractive and/or interference effects may be provided by one or more masks located downstream of a radiation source and having selected patterns for shaping the emitted radiation, by shaping of the actual radiation source to cause interference in the emitted radiation generating selected focusing spot at a selected distance, or by shaping radiation emission from an emitting material but selective spatial emission from a radiation source.

More specifically, in some configurations, the radiation emitting elements include one or more radiation sources and a radiation focusing element located in path of radiation remitted from said one or more radiation sources. The radiation focusing elements may be diffractive elements configured for directing emitted radiation to be focused at said corresponding select spot on said aperture plane. For example, the radiation focusing element may be a zone plate. In some additional configurations, the radiation emitting element may further comprise a metallic radiation absorbing plate with selected channels of transmission of radiation therethrough. The channels may be drilled through the radiation absorbing plate for reducing leakage of radiation and directing high radiation intensity toward selected diffraction of orders providing efficient focusing of the emitted radiation onto the selected focusing spot.

In some further configurations, the radiation emitting elements may be shaped to provided structure radiation emission pattern directing the emitted radiation toward a selected focusing spot on the aperture plane. The radiation emitting element may be configures with spiral structure having period variation with respect to radiation axis thereof to thereby direct large portion of the emitted radiation in converging path toward a focusing spot located at a selected distance. The distance of the focusing spot and dimensions thereof being determined by diameter of the radiation emitting element and variations in periodicity of the spiral structure thereof.

In some additional configurations, the radiation emitting element may be configured with a radiation source (e.g. tungsten plate) configured for emitting radiation in response to electrons impinging thereon, and electron emitting tube (e.g. electron gun) configured for selectively direct emitted electrons of selected energy onto the radiation source. The electron emitting tube is configured for directing electrons to selected locations on the radiation source/plate to thereby generate selected spatial radiation emission pattern. The pattern may be selected to provide interference relations resulting in focusing of the emitted radiation onto the selected focusing spot.

Generally, the use of non-optical wavelength such as X-ray or gamma limits image optimizing techniques that are suitable for optical wavelength. The use of pinhole imaging, and the technique described herein, allows additional imaging optimizing techniques enabling super-resolution image acquisition. For example, the present technique may utilize two or more grating located at selected planes within the imaging system, enabling super resolution imaging as described in Z. Zalevsky et al “Super resolution optical systems using fixed gratings,” Opt. Commun. 163, 79-85 (1999) as well as in A. Shemer et al “Time multiplexing super resolution based on interference grating projection,” Appl. Opt. 41, 7397-7404 (2002), incorporated herein by reference in connection with super resolution techniques. Such super resolution techniques have been previously described in connection with optical imaging. More specifically, in such configurations the imaging system may further comprise at least two gratings (e.g. Dammann gratings having phase or amplitude affecting pattern with selected predetermined period) located in selected planes, where at least one grating is located between the object being imaged and the aperture plane, and at least one grating is located between the aperture plane and the detector array. The use of properly located grating having selected spatial period enable creation of super-resolved image at the detection plane. In some configurations, the gratings may be static to provide field of view multiplexing super resolution. In some other configurations, the system may include one or two grating elements, generally one grating element for encoding radiation filed and one other grating element for decoding the radiation field (in which case, the decoding grating may be physical or digital grating), that are moveable during period of image acquisition. Generally, moveable gratings provide time multiplexing super resolution. More specifically, the two grating elements may be moveable for each period of image acquisition associated with a corresponding set of radiation emitting elements (associated with set of apertures). In some other configurations, the system may include three or more grating elements that may be stationary.

In some configurations enables enhanced transmission of high spatial frequencies maintaining the high-resolution image data, while not limiting transmission of low spatial frequency information. To this end, the gratings are located at predetermined locations and at predetermined distances therebetween. Each grating performs modulation/encoding on the radiation emitted from the plurality of emitting elements to thereby adapt the object's signal to the channel capacity.

Thus, according to a broad aspect, the present invention provides an imaging system comprising:

a plurality of radiation emitting elements, configured for emitting wave or wave-like (e.g. electromagnetic or ultra-sonic) radiation of a selected wavelength range, each of said radiation emitting elements is configured for focusing said radiation onto corresponding selected spot on an aperture plane of the imaging system; and

a detector array located at a selected image plane located downstream of said aperture plane;

said imaging system is configured for imaging an object located between said plurality of radiation emitting elements and said aperture plane.

Typically for ultra-sonic radiation, the radiation emitting elements may comprise one or more ultra-sonic transducers. Alternatively, when used with electromagnetic radiation, the present technique is advantageous as compared to alternative techniques using non-optical electromagnetic radiation such as UV, X-ray, Gamma etc.

According to some embodiments, said plurality of radiation emitting elements comprises a selected number of groups of radiation emitting elements, each group containing one or more radiation emitting elements, wherein each group is oriented for directing radiation emitted therefrom toward a common selected spot on said aperture plane. The plurality of radiation emitting elements may be arranged on a sphere with respect to the aperture plane.

According to some embodiments, the system may further comprise an aperture mask located at said aperture plane and comprising a plurality of apertures associated with plurality of selected spots associated with said plurality of radiation emitting elements.

According to some embodiments, said plurality of radiation emitting elements comprise one or more radiation emitting elements comprising a radiation source and a radiation focusing element located in path of radiation remitted from said radiation source, thereby directing emitted radiation to be focused at said corresponding select spot on said aperture plane. The radiation focusing element may be one or more diffractive element. The radiation focusing element may be one or more zone plate.

According to some embodiments, plurality of radiation emitting elements comprise one or more radiation emitting elements formed with a selected radiation emitting pattern causing emitted radiation to propagate to be focused at said corresponding select spot on said aperture plane. The selected radiation emitting pattern may be in the form of spiral rolled radiation emitting material having density reduced toward center thereof.

According to some embodiments, said plurality of radiation emitting elements comprise one or more controllable radiation emitting elements formed with a radiation emitting material and corresponding electron emitting unit configured for directing electrons at said emitting material for causing said emitting material to emit electromagnetic radiation, said electron emitting unit is controllably configured for providing spatial emitting pattern enabling focusing of said electromagnetic radiation.

According to some embodiments, said plurality of radiation emitting elements further comprising corresponding plurality of drilled plates having tunnels allowing radiation to propagate in converging path toward the corresponding focusing spots.

According to some embodiments, said one or more controllable radiation emitting elements configured to provide structure radiation pattern for providing virtual grating in pattern of emitted radiation, the system further comprises at least one additional moveable grating located between said aperture plane and detector array to thereby provide super resolution imaging.

According to some embodiments, the system may further comprise at least first and second grating elements, said first grating element being located at selected plane between location of said object and said aperture plane, and said second grating element being located between said aperture plane and the detector array.

The at least first grating elements may be moveable with respect to general axis of radiation propagation within time of image acquisition. In some embodiments, the first grating element may be located immediately after location of said object in path of radiation propagation direction and is moveable in time to provide time multiplexing encoding of the radiation, said second grating element is located between said aperture plane and the detector array and is configured for decoding said time multiplexing encoding to provide super-resolution in image collection. In some embodiments, the second grating may be virtual grating provided by readout pattern of the detector array.

Additionally or alternatively, the system may further comprise at least third static grating element located between said second grating element and said detector array. Generally, periods and location of the first, second and third gratings satisfy required mathematical conditions to obtain a field of view multiplexing super resolved reconstruction. In some configurations, periods and location of the first, second and third gratings may be selected to satisfy:

mv₁+nv₂+lv₃=0 i)

z₁mv₁−z₂nv₂−z₃lv₃=0 ii)

λz₁mv₁/2−z₂λmnv₁v₂−z₂λn²v₂²/2−z₃λmlv₁v₃−z₃λl²v₃²/2−z₃λnlv₂v₃=N iii)

where v₁, v₂ and v₃ are respective periods of the first, second and third gratings, z₁ is distance between the first grating and the object, z₂ and z₃ are respective distances between the second and third gratings and the detector array, is wavelength of the radiation used, m, n, l and N are integers. For example, periods of the first and third grating elements may be equal, and period of the second grating element is twice the period of the first grating element.

According to one other broad aspect, the present invention provides a controllable radiation emitting unit comprising radiation emitting material and corresponding electron emitting unit, said electron emitting unit being configured for selectively directing electrons at said emitting material for causing said emitting material to emit electromagnetic radiation.

According to yet another broad aspect, the present invention provides a radiation emitting unit configured in spiral pattern thereby focusing emitted radiation to a selected focusing spot.

According to one other broad aspect, the present invention provides a method of use in imaging with non-optical radiation, the method comprising:

providing a plurality comprising a selected number of groups of radiation emitting elements, each radiation emitting element being configured for emitting electromagnetic radiation of a selected wavelength range and for focusing said radiation onto corresponding selected spot on a selected aperture plane;

radiation emitting elements of each group are oriented for directing radiation emitted therefrom toward a common selected spot on said aperture plane, where radiation emitting element of different group are oriented toward different selected spots;

sequentially operating selected sets of said groups of radiation emitting element for emitting radiation for selected periods, and detecting radiation at a detection plane;

processing the detected radiation using data of arrangement of the focusing spots and said selected periods for reconstruction of image of an object.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 illustrates schematically an imaging system according to some embodiments of the present invention;

FIG. 2 schematically illustrates an additional configuration of an imaging system according to some embodiments of the invention;

FIG. 3 shows one possible configuration of a radiation emitting elements utilizing radiation focusing unit according to some embodiments of the invention;

FIG. 4 shows one other configuration of a radiation emitting element utilizing shaped radiation emitting structure according to some embodiments of the invention;

FIG. 5 shows an additional configuration of radiation emitting element utilizing selected emission pattern according to some embodiments of the invention;

FIG. 6 illustrates one additional radiation emitting unit configured as vacuum tube having multiple radiation emitting cathode enabling structure radiation according to some embodiments of the present invention;

FIG. 7 schematically illustrates radiation converger element configured as a radiation absorbing mask having plurality of tunnels through the mask, where the tunnels allow radiation to propagate in selected paths according to some embodiments of the present invention;

FIG. 8 schematically illustrates an imaging system configured for field of view multiplexing super resolution imaging of X-ray, gamma, or other non-optical radiation according to some embodiments of the invention; and

FIG. 9 schematically illustrates an imaging system configured for time multiplexing super resolution imaging of X-ray, gamma, or other non-optical radiation according to some embodiments of the invention.

### DETAILED DESCRIPTION OF EMBODIMENTS

As indicated above, the present invention provides a system and a technique enabling imaging using optical as well as non-optical radiation. In various configurations, the present technique provides advantageous imaging performance as compared to conventional X-ray imaging techniques providing high resolution and high energetic efficiency for imaging selected objects. To this end, the present technique utilizes the concept of pinhole imaging, and imaging through a selected set of pinhole arrays, enabling imaging without refractive optical elements while maintaining high efficiency in terms of brightness and contrast. In this connection it should be noted that the present technique is suitable for imaging with optical and non-optical wavelength ranges and is specifically advantageous for imaging with non-optical wavelength ranges. Such non-optical wavelength ranges may include X-ray, Gamma, UV, as well as ultra-sound and other wave-like radiation. It should also be understood that the present application describes the use of X-ray and/or Gamma radiation for simplicity and these terms should be understood broadly as referring to non-optical wavelength ranges suitable for any selected application.

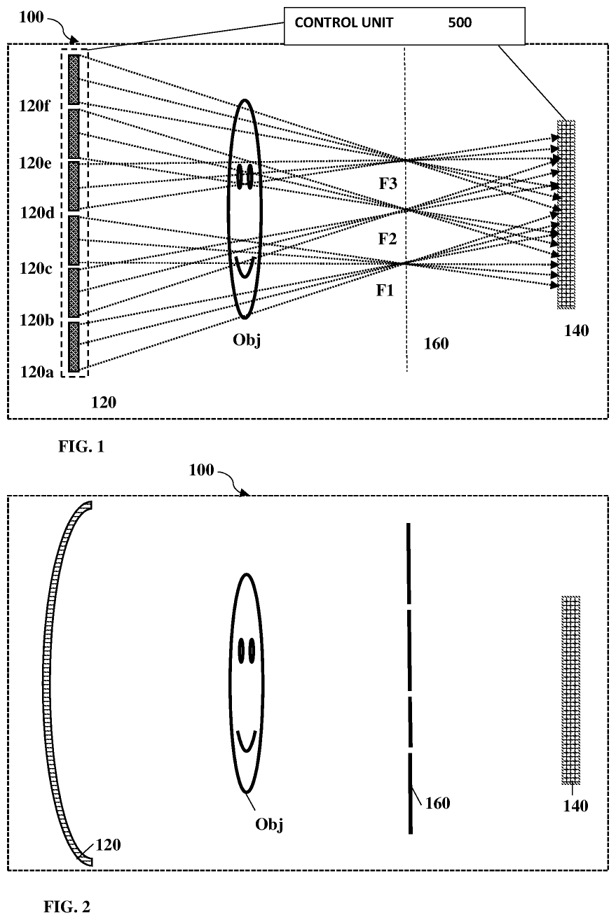

Reference is made to FIG. 1 schematically illustrating an imaging system 100 according to some embodiments of the invention. The imaging system 100 includes a radiation source unit 120 including a plurality of radiation emitting elements (six such emitting elements 120a-120f are exemplified, where two or more such emitting elements may preferably be used) and a detector array 140. The system 100 may also include a sample mount/holder located at a selected location between the radiation source unit 120 and the detector array 140, the sample mount is represented in FIG. 1 by location of the object Obj being imaged. Generally, the system includes an aperture plane 160 located downstream of the object mount with respect to direction of radiation propagation, where a mask unit may be located. The aperture plane defines location of one or more pinhole array and defined by propagation of radiation rays between the radiation source unit 120 and the detector array 140. It should be noted that an actual mask unit having one or more pinhole arrays may or may not be placed at the aperture array in accordance with certain embodiments of the present technique.

The radiation emitting elements 120a-120f are each configured for emitting radiation in one or more selected wavelength ranges (e.g. X-ray) in response to operational command (e.g. electrical current). The radiation emitting elements 120a-120f are further configured for emitting radiation in converging path directed at corresponding focusing spots, marked as F1-F3, on the aperture plane. Generally, the plurality of radiation emitting elements may include one or more groups of radiation emitting elements, where each group of radiation emitting elements are oriented and configured for directing emitted radiation to a common focusing spot on the aperture plane. For example, radiation emitted from elements 120a and 120c is directed toward focusing spot F1, radiation emitted from elements 120b and 120e is directed toward focusing spot F2, and radiation emitted from elements 120d and 120f is directed toward focusing spot F3. Thus, radiation emitted from one or more radiation emitting elements may be transmitted through at least a portion of the object and radiation components further transmitted through the object pass through the focusing spot and impinge on the detector array, thereby providing image of the relevant portion of the object formed by pinhole imagine technique.

Generally, the plurality of radiation emitting elements 120a-120f are separated into plurality of groups of radiation emitting elements as indicated above. Each of the plurality of groups of radiation emitting elements directs emitted radiation toward a common focusing spot. the plurality of groups generally includes two or more sets of groups, where groups of each set direct emitted radiation toward a selected arrangement of focusing spots forming an effective aperture array on the aperture plane.

This arrangement enables the imaging system 100 to provide imaging of the object by directing radiation toward two or more arrays of focusing spots and collecting corresponding image data associated with two or more collected patterns, each collected pattern includes an arrangement of one or more image replications associated with the one or more focusing spots of the array. Selection of the spatial arrangement of the focusing spots for groups of radiation emitting elements associated with each set, and reconstructions of the collected image data is described in U.S. Pat. No. 10,033,996 indicating details of spatial arrangement of the aperture arrays (associated with arrangement of focusing spots in the present technique) and with respect to reconstructions of collected image data to provide two- and three-dimensional image reconstructions.

More specifically location of the aperture plane 160 may be determined, as well as a desired energy transmission. The energy transmission may be determined by selection of number of focusing spots, and/or in accordance with number of radiation emitting elements directed at each spot. The total number of aperture arrays is selected (e.g. three or more), where additional aperture arrays may enhance complexity but allow increased tailoring of the transmission function. The arrangement of the focusing spots in a first array are selected. It should be noted that the order of selection of the arrays is of no importance at the imaging session. Generally, the arrangement of focusing spots of the first array may be determined arbitrarily, however generally a simple arrangement of one spot at the center of the radiation collection surface and one focusing spot at certain distance therefrom along a selected axis may be preferred. Generalization to two dimensional arrangements may be done by copying 1-dimensional arrangement along a second axis and/or rotation of such 1-dimensional arrangement.

Once a first array of focusing spots is selected, the corresponding effective transmission function is determined, and the “problematic” spatial frequencies are marked. As indicated above, the effective transmission function is determined in accordance with

$$F ( u , v ) = {\sum }_{n = 1}^{N} {\sum }_{m = 1}^{N} {e}^{- 2 π i ( {ud}_{n}^{( x )} + {vd}_{m}^{( y )} )}$$

where dₙ^{(x)}, dₘ^{(y)} are the (x,y) coordinated of the locations of the focusing spots within the array relative to the center of the array, u and v are spatial frequencies, and N is the number of focusing spots.

The “problematic” spatial frequencies that satisfy F^{(l)}(u₁, v₁)=0 for a specific array are preferably marked. As well as spatial frequencies for which the transmission is under a predetermined threshold (e.g. below 0.1). Such spatial frequencies are marked only within the resolution limits defined by the aperture diameter. Based on these marked spatial frequencies, additional arrays of focusing spots are determined. The number of spots is selected in accordance with desired resolution and energy transmission, while the arrangement of the focusing spots is determined to provide finite, and preferably high, transmission values of the corresponding effective transmission function for the spatial frequencies marked for the previous array(s). This process may be performed for two, three or more arrays until an appropriate set of focusing spots is selected.

The selection of an appropriate set of array of focusing spots may be directed at optimizing the transmission of the arrays for selected spatial frequencies. To this end, the selection process may also include determining an estimated total effective transmission function, assuming equal exposure times for all arrays. The estimated effective transmission function may then be compared to a transmission function formed by single arrangement of radiating elements directed to form a single focusing spot. Generally, the set of arrays is selected to optimize transmission of spatial frequencies with the resolution limits to thereby optimize imaging of the region of interest. To this end the aperture arrays, as well as corresponding exposure times are selected such that for at least some spatial frequencies within the desired resolution limits, the total effective transmission function provides transmission that is greater than that of the single arrangement transmission function.

The collected image data is reconstructed by determining spatial frequencies in accordance with the transmission functions and converting to spatial coordinates for obtaining image data.

Generally, the system 100 may also include, or be associated with, a control unit 500. The control unit 500 is connectable to the radiation source unit 120 for selectively operating one or more groups of radiation emitting elements, in accordance with sets of radiation emitting elements, and for receiving collected image data from the detector array 140. In some configurations, the control unit may also be configured for processing the collected image data for reconstructions of two- or three-dimensional image of the object.

As indicated above, the present technique utilizes concepts of pinhole imaging by directing emitted radiation toward one or more focusing spots located at a selected aperture plane. In this connection, the system 100 may or may not include physical mask located at the aperture plane. FIG. 2 exemplifies an arrangement of system 100 including aperture array mask 160 located at the aperture plane. The mask 160 typically include an arrangement of apertures of selected dimensions, corresponding to the arrangement of the focusing spots associated with the plurality of radiation emitting elements 120a-120f. More specifically, the mask 160 may include an arrangement of apertures associated with all the focusing spots formed by the radiation emitting elements, or include a replaceable arrangement of two or more masks, each carrying an aperture array associated with array of focusing spots formed by a corresponding set of radiation emitting elements.

As also exemplified in FIG. 2, the radiation source unit 120 including a plurality of radiation emitting elements, may be configured with spherical arrangement with respect to a central point located on or in vicinity to the aperture plane 160. Accordingly, the radiation emitting elements are arranged, in spatially separated arrangement on a shell of a portion of a sphere. This configuration provides substantially similar distance between the radiation emitting elements 120a-120f and the corresponding focusing spots allowing the use of similar radiation emitting elements while varying orientation thereof for providing the selected arrangement of focusing spots of the aperture plane 160. It should be noted that although exemplified together in FIG. 2, the use of aperture mask on aperture plane 160 and spherical arrangement of the radiation source unit 120 are separate features of the present technique that may be used independently, separately or together in different embodiments of the invention.

As indicated above, the present invention may provide imaging using non-optical frequency ranges. Such non-optical frequencies may generally include X-ray, Gamma and other non-optical frequencies as the case may be. Accordingly, the present technique provides a radiation emitting element configured for emitting radiation directed at a selected focusing spot located at a selected distance therefrom. To this end, the present technique utilizes diffractive and interference effects directing remitted radiation in converging paths toward the selected focusing spots.

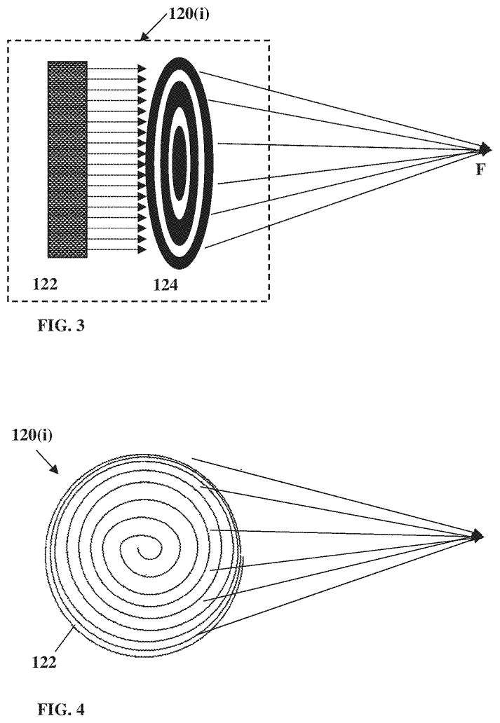

Reference is made to FIG. 3 exemplifying a radiation emitting element 120(i) according to some embodiments of the invention. The radiation emitting element 120(i), in this example includes a radiation source 122 configured for emitting radiation (e.g. x-ray radiation) and a radiation shaping element 124 having a spatial amplitude and/or phase varying pattern. The radiation shaping element 124 may generally be some diffractive elements and is located in an optical path of the radiation emitted from the radiation source 122. Pattern of the radiation shaping elements is selected to cause diffraction of radiation passing therethrough thereby for directing at least a portion of the radiation in a converging path toward a selected focusing spot F. In this connection the radiation shaping element 124 may be configured as zone plate element having circular arrangement of transmitting and blocking regions. The arrangement of the pattern of the radiation shaping element 124 is preferably configured for directing radiation towards a single selected diffraction order, e.g. zero order for providing high intensity at the selected focusing spot and optimizing efficiency of imaging.

An additional example of radiation emitting element 120(i) configured for directing emitted radiation toward selected focusing spot is exemplified in FIG. 4. In this example, the radiation source is shaped to provide emission of structured radiation in converging path toward the focusing spot. To this end, radioactive material (e.g. Cobalt or Americium) is used for coating a carrying wire and shaped to provide spatial distribution of the radiation (x-ray) source 122. For example, the radiation source may be arranged in spatial configuration as exemplified in FIG. 4 having variation of spatial period corresponding to zone plate arrangement. More specifically, the period of the spiral arrangement is shorter at periphery of the radiation source and longer at center thereof. This radiation emitting element 120(i) may preferably be used with one or more collimators located in path of emitted radiation for directing the greater radiation intensity emitted from periphery of the radiation source in a converging path toward the selected focusing spot (selected in accordance with orientation and period variation of the radiation source).

In some additional configurations, the radiation emitting elements may be configured to provides structured radiation emission using selective impinging of electrons on radiation emitting plate (e.g. tungsten). FIG. 5 exemplifies a radiation emitting element 120(i) including electron source 13, vacuum tube 14 in which electrons emitted from the electro source are propagating, and radiation source 122, e.g. cathode, configured for emitting radiation in response to electrons impinging thereon. The electrons emitted from the source 13 are selectively directed toward selected regions of the emitting cathode (radiation source 122) using electric field applied between metallic plates 15 or using variation in magnetic field applied in direction of propagation of the electrons, similar to direction of electrons in CRT tubes as known in the art. The electron tube is operated for providing controllable pattern of radiation emitted from the radiation source 122, enabling emission of radiation in converging path therefrom. In some configurations, the radiation source 122 (e.g. cathode, plate) may be curved to form a portion of a sphere, thereby providing improved converging distribution of emitted radiation.

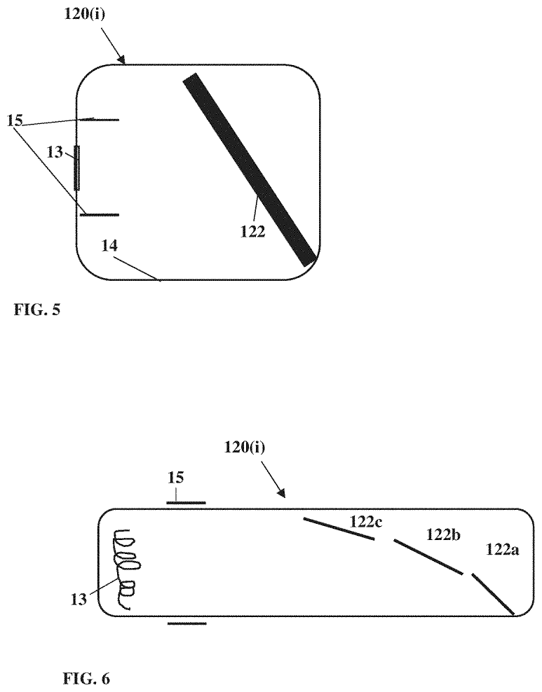

In additional configurations, as exemplified in FIG. 6, the radiation emitting element 120(i) may be configured from a vacuum tube including two or more radiation sources, e.g. 122a to 122c may be formed as radiation emitting cathodes, arranges with selected angular orientation to directing emitted radiation toward the selected focusing spot. Selective operation of the different cathode elements enables focusing of the emitted radiation to the selected focusing spot in a converging path.

Such radiation emitting elements capable of providing patterned/structured radiation may be tuned for providing virtual grating in pattern of emitted radiation. Such virtual grating pattern may be moveable during period of image acquisition as described further below.

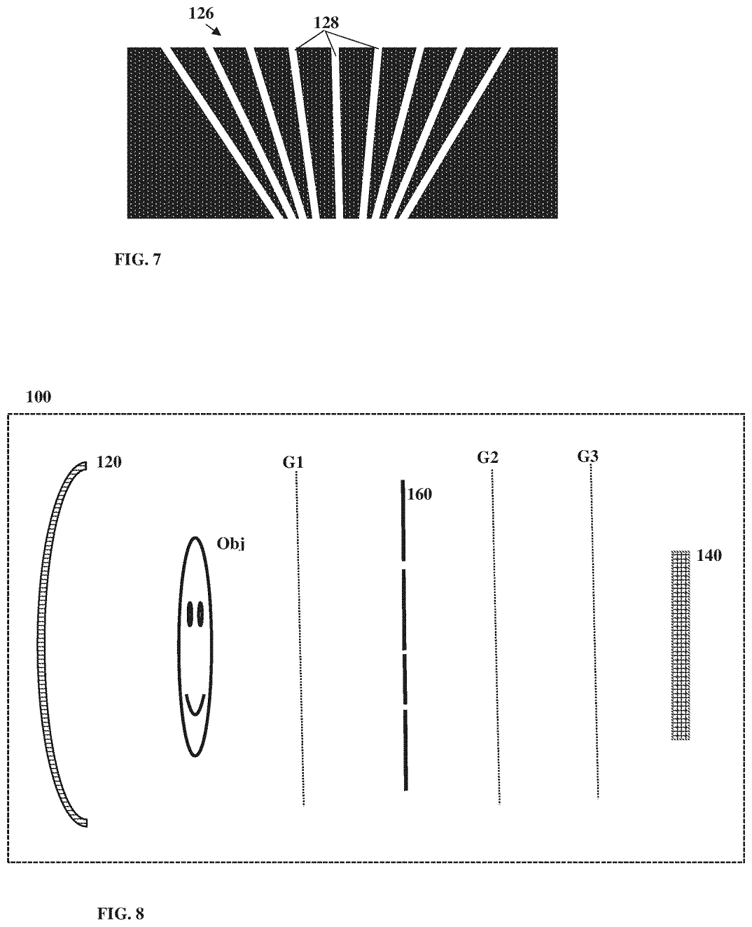

Reference is made to FIG. 7 illustrating schematically radiation converger elements 126. The radiation converger element 126 is formed by a thick plate made of radiation absorbing material (e.g. Lead Pb) having a plurality of holes 128 drilled through the plate. The holes 128 are drilled in a converging path allowing radiation to propagate in the selected converging path. The radiation converger 126 may be used in combination with any radiation emitting element, and specifically with the radiation emitting elements exemplified herein in FIGS. 3 to 6 for improving resolution.

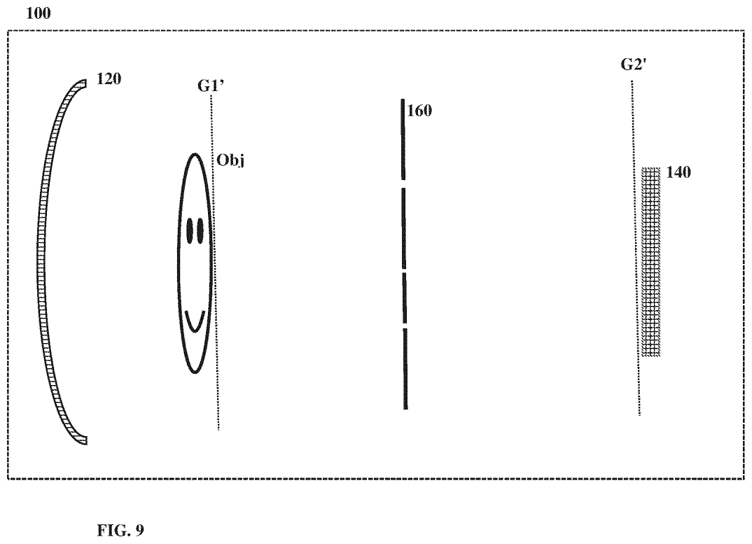

Reference is made to FIGS. 8 and 9 illustrating an additional configuration of imaging system 100, utilizing selected field of view multiplexing (FIG. 8) and time multiplexing (FIG. 9) super resolution imaging techniques. As indicated above, generally non-optical wavelengths, such as X-ray, provide limits imaging capabilities due to the limited radiation manipulation technique reducing the ability to use refraction of radiation. The use of pinhole imaging, and the above described technique, allows additional imaging optimizing techniques enabling super-resolution image acquisition. For example, the present technique may utilize two or more grating located at selected planes within the imaging system, enabling super resolution imaging as described in Z. Zalevsky et al “Super resolution optical systems using fixed gratings,” Opt. Commun. 163, 79-85 (1999) as well as in A. Shemer et al “Time multiplexing super resolution based on interference grating projection,” Appl. Opt. 41, 7397-7404 (2002), incorporated herein by reference in connection with super resolution techniques.

In this connection, FIG. 8 illustrates an imaging system 100 including two or more grating elements, three such gratings are exemplified as G1, G2 and G3. The gratings have periods selected to allow transmission of the used radiation wavelength ranges and avoid distortion of spatial frequency spectrum. The grating G1, G2 and G3 are located between the object Obj and the detector array 140 of the system 100 at a predetermined distance there between, in an optical path of the radiation emitted from the radiation source 120. More specifically, grating G1 is located downstream of the object Obj, between the object Obj and the aperture plane 160. Gratings G2 and G3 are located between the aperture plane 160 and the detector array 140. Generally, the first, second and third gratings satisfy required mathematical conditions to obtain a field of view multiplexing super resolved reconstruction. Such mathematical conditions related to periods and location of the first, second and third gratings. Specifically, the mathematical condition may be indicated by:

mv₁+nv₂+lv₃=0 i)

z₁mv₁−z₂nv₂−z₃lv₃=0 ii)

λz₁mv₁/2−z₂λmnv₁v₂−z₂λn²v₂²/2−z₃λmlv₁v₃−z₃λl²v₃²/2−z₃λnlv₂v₃=N iii)

where v₁, v₂ and v₃ are respective periods of the first, second and third gratings, z₁ is distance between the first grating and the object, z₂ and z₃ are respective distances between the second and third gratings and the detector array, is wavelength of the radiation used, m, n, l and N are integers. For example, the periods of the gratings may be selected such that the periods of gratings G1 and G3 are similar and the period of grating G2 may be twice the period of G1.

In some configurations, as exemplified in FIG. 9, two grating elements (e.g. G1′ and G2′) may be used. At least one grating element G1′ is located immediately after location of the object Obj and is moveable with respect to the object for encoding of the radiation filed. Another grating element G2′ may be physical grating or used as virtual grating by digital sampling of detector data located close to (or on) the detector array 160 for decoding the radiation filed to provide time multiplexing super-resolution. The grating elements are configured to be moveable with respect to general axis of radiation propagation (i.e. perpendicular to the general axis) within period of image acquisition. This allows averaging super resolution by providing synthetic aperture acting as smaller pinholes (as opposed to larger lens aperture used in imaging of optical wavelengths). Generally, the moveable gratings G1′ and G2′ may be repeatedly moved for each period of image acquisition associated with sets of apertures as described herein above. In some additional configurations, the first grating element G1′ may be replaced by virtual grating formed by structured radiation pattern, e.g. emitted by radiation emitting element such as described in FIG. 5 or 6. In some other configurations, the second grating G2′ may be virtual and generated by readout pattern of the detector array 160.

Generally, in the field of medical x-ray imaging, it is desired to reduce energy of ionizing radiation irradiated on body of a patient. The present technique enables high resolution imaging with improved energetic efficiency, and thereby enable providing improved imaging of patient, or a portion of body of a patient, with lower radiation intensity.

1. An imaging system comprising:
a radiation emitting unit, configured for emitting wave radiation of a selected wavelength range and comprising a plurality of radiation emitting elements, each of said plurality of radiation emitting elements is configured for focusing said radiation onto corresponding selected spot on an aperture plane of the imaging system, forming a selected arrangement of focusing spots; and
a detector array located at a selected image plane located downstream of said aperture plane;
said imaging system is configured for imaging an object located between said plurality of radiation emitting elements and said aperture plane.

2. The system of claim 1, wherein said wave radiation is electromagnetic radiation having non-optical wavelength.

3. The system of claim 1 wherein said wave radiation is ultra-sonic acoustic radiation, said radiation emitting elements comprise ultra-sonic transducers.

4. The system of claim 1, wherein said plurality of radiation emitting elements has at least one of the following configurations: (i) comprises a selected number of groups of radiation emitting elements, each group containing one or more radiation emitting elements, wherein each group is oriented for directing radiation emitted therefrom toward a common selected spot on said aperture plane; (ii) is arranged on a sphere with respect to the aperture plane; (iii) comprises one or more radiation emitting elements comprising a radiation source and a radiation focusing element located in path of radiation remitted from said radiation source, thereby directing emitted radiation to be focused at said corresponding select spot on said aperture plane.

5. (canceled)

6. The system of claim 1, further comprising an aperture mask located at said aperture plane and comprising a plurality of apertures associated with plurality of selected spots associated with said plurality of radiation emitting elements.

7. The system of claim 1, wherein said plurality of radiation emitting elements comprise one or more radiation emitting elements comprising a radiation source and a radiation focusing element located in path of radiation remitted from said radiation source, thereby directing emitted radiation to be focused at said corresponding select spot on said aperture plane, wherein said radiation focusing element is configured as a diffractive element or a zone plate.

8-9. (canceled)

10. The system of claim 1, wherein said plurality of radiation emitting elements comprise one or more radiation emitting elements formed with a selected radiation emitting pattern causing emitted radiation to propagate to be focused at said corresponding select spot on said aperture plane.

11. The system of claim 10, wherein said selected radiation emitting pattern is in the form of spiral rolled radiation emitting material having density reduced toward center thereof.

12. The system of claim 1, wherein said plurality of radiation emitting elements comprise one or more controllable radiation emitting elements formed with a radiation emitting material and corresponding electron emitting unit configured for directing electrons at said emitting material for causing said emitting material to emit electromagnetic radiation, said electron emitting unit is controllably configured for providing spatial emitting pattern enabling focusing of said electromagnetic radiation.

13. The system of claim 1, wherein said plurality of radiation emitting elements further comprising corresponding plurality of drilled plates having tunnels allowing radiation to propagate in converging path toward the corresponding focusing spots.

14. The system of claim 12, wherein said one or more controllable radiation emitting elements configured to provide structure radiation pattern for providing virtual grating in pattern of emitted radiation, the system further comprising at least one additional moveable grating located between said aperture plane and detector array to thereby provide super resolution imaging.

15. The system of claim 1, further comprising at least first and second grating elements, said first grating element being located at selected plane between location of said object and said aperture plane, and said second grating element being located between said aperture plane and the detector array.

16. The system of claim 15, wherein said at least first and second grating elements are moveable with respect to general axis of radiation propagation within time of image acquisition.

17. The system of claim 16, characterized by at least one of the following: (a) said first grating element is located immediately after location of said object in path of radiation propagation direction and is moveable in time to provide time multiplexing encoding of the radiation, said second grating element is located between said aperture plane and the detector array and is configured for decoding said time multiplexing encoding to provide super-resolution in image collection; (b) the second grating is virtual grating provided by readout pattern of the detector array.

18. (canceled)

19. The system of claim 15, further comprising at least third grating element located between said second grating element and said detector array.

20. The system of claim 19, characterized by at least one of the following configurations: (1) periods and location of the first, second and third gratings satisfy required mathematical conditions to obtain a field of view multiplexing super resolved reconstruction; (2) periods of the first and third grating elements are equal, and period of the second grating element is twice the period of the first grating element.

21. (canceled)

22. A controllable radiation emitting unit comprising radiation emitting material and corresponding electron emitting unit, said electron emitting unit being configured for selectively directing electrons at said emitting material for causing said emitting material to emit electromagnetic radiation.

23. A radiation emitting unit configured in spiral pattern thereby focusing emitted radiation to a selected focusing spot.

24. A method of use in imaging with non-optical radiation, the method comprising:
providing a plurality comprising a selected number of groups of radiation emitting elements, each radiation emitting element being configured for emitting electromagnetic radiation of a selected wavelength range and for focusing said radiation onto corresponding selected spot on a selected aperture plane;
radiation emitting elements of each group are oriented for directing radiation emitted therefrom toward a common selected spot on said aperture plane, where radiation emitting element of different group are oriented toward different selected spots;
sequentially operating selected sets of said groups of radiation emitting element for emitting radiation for selected periods, and detecting radiation at a detection plane;
processing the detected radiation using data of arrangement of the focusing spots and said selected periods for reconstruction of image of an object.

25. An imaging system comprising:
a radiation emitting unit, configured for emitting wave radiation of a selected wavelength range and comprising a plurality of radiation emitting elements, each of said plurality of radiation emitting elements is configured for focusing said radiation onto corresponding selected spot on an aperture plane of the imaging system, forming a selected arrangement of focusing spots, wherein said plurality of radiation emitting elements comprises one or more radiation emitting elements formed with a selected radiation emitting pattern in the form of spiral rolled radiation emitting material having density reduced toward center thereof causing emitted radiation to propagate to be focused at said corresponding selected spot on said aperture plane; and
a detector array located at a selected image plane located downstream of said aperture plane;
said imaging system is configured for imaging an object located between said plurality of radiation emitting elements and said aperture plane.
